(19) United States
(12) Patent Application Publication
(10) Pub. No.: US 2021/0380706 A1
(43) Pub. Date: Dec. 9, 2021

(54) METHODS FOR TREATING SEVERE ASTHMA IN PATIENTS WITH NASAL POLYPOSIS

(71) Applicant: ASTRAZENECA AB, Sodertalje (SE)

(72) Inventors: Maria Esther GARCIA GIL, Barcelona (ES); James ZANGRILLI, Gaithersburg, MD (US); Anne Burden, Cambridge (GB); James KREINDLER, Gaithersburg, MD (US)

(21) Appl. No.: 17/338,869

(22) Filed: Jun. 4, 2021

Related U.S. Application Data
(60) Provisional application No. 63/035,021, filed on Jun. 5, 2020.
(60) Provisional application No. 62/706,245, filed on Aug. 6, 2020.
(60) Provisional application No. 63/112,919, filed on Nov. 12, 2020.

(51) Int. Cl. C07K 16/28; A61P 11/06

(52) U.S. Cl. CPC C07K 16/2866; A61K 2039/505; A61P 11/06

(57) ABSTRACT

Provided herein are methods of reducing exacerbations of asthma in an asthma patient with nasal polyposis, comprising administering to the patient an effective amount of the anti-interleukin-5 receptor (IL-5R) antibody benralizumab or an antigen-binding fragment thereof.

### BACKGROUND

More than 300 million people around the world have asthma. Despite the use of long-acting bronchodilators and inhaled corticosteroids, unscheduled visits to doctor offices, visits to emergency departments (ED), and hospitalizations due to asthma exacerbations occur frequently and account for a significant proportion of healthcare costs attributable to asthma. (Masoli M, et al. Allergy 59: 469-78(2004)).

Relapse following acute asthma exacerbation has been reported to range from 41 to 52% at 12 weeks despite the use of systemic steroids upon discharge (Lederle F, et al. Arch Int Med 147:2201-03 (1987)). Management of these patients has proved problematic due either to severe refractory disease or inability and/or unwillingness to comply with medical treatment. In one study of patients admitted to the hospital, some with near fatal asthma, 50% were non-compliant with systemic corticosteroids at 7 days following discharge (Krishnan J, et al. AJRCCM 170: 1281-85 (2004)). Many factors may contribute to non-compliance including poor access to routine quality healthcare (particularly in the inner city), lack of education or understanding of their disease, unwillingness to accept the chronic nature of their disease, or inability to obtain medications.

Many lines of evidence implicate eosinophils as one of the main causative cells of asthmatic airway inflammation (James A. Curr Opin Pulm Med 11(1):1-6 (2005)). Peripheral blood (PB) eosinophilia is a risk factor for relapse of acute asthma (Janson C and Herala M. Resp Med 86(2):101-104 (1992)). In subjects with peripheral blood eosinophilia, the risk of dying from asthma was 7.4 (confidence interval, 2.8-19.7) times greater than in those without eosinophilia (Ulrik C and Fredericksen J. Chest 108:10-15 (1995)). Necropsy results have identified 2 distinct pathogenic inflammatory mechanisms of fatal asthma (Restrepo R and Peters J. Curr Opin Pulm Med 14: 13-23 (2008)). A neutrophilic infiltrate is more prominent in those dying suddenly (approximately within 2 hours on onset of symptoms), while an eosinophilic infiltrate is more common in those dying from more protracted asthma crises. Sputum and blood eosinophils can also be increased in patients presenting to the ED with rapid onset of asthma symptoms (Bellido-Casado J, et al. Arch Bronconeumol 46(11): 587-93 (2010)). Therapies that target eosinophils lead to a reduction in the number and severity of asthma exacerbations as compared to the use of clinical guidelines (Green R, et al. Lancet 360:1715-21 (2002); Haldar P, et al. NEJM 360:973-84 (2009)).

Approximately 60% of patients with chronic rhinosinusitis with nasal polyposis (NP) have asthma, with the frequency of NP increasing with advancing age. Patients with chronic rhinosinusitis with NP and severe and steroid-resistant asthma have reduced asthma control and a high level of disease burden, which negatively impacts health-related quality of life (HRQOL). NP is often associated with severe and steroid-resistant asthma, with increased blood eosinophil counts (BEC), airway obstruction, inflammatory cells, as well as reduced asthma control for patients with asthma along with NP compared with those without NP. Therefore, the combination of asthma and NP provides significant treatment challenges and substantial disease burden, along with a significantly greater number of asthma exacerbations per year, which negatively impacts health-related quality of life (HRQOL), thereby necessitating novel therapies for better patient outcomes.

Benralizumab (MEDI-563) is a humanized monoclonal antibody (mAb) that binds to the alpha chain of the interleukin-5 receptor alpha (IL-5Rα), which is expressed on eosinophils and basophils. It induces apoptosis of these cells via antibody-dependent cell cytotoxicity. A single intravenous (IV) dose of benralizumab administered to adults with mild asthma provoked prolonged PB eosinopenia likely due to the effects on eosinophil/basophil bone marrow progenitors that express the target (Busse W, et al. JACI 125: 1237-1244 e2 (2010)). In addition, a single dose of benralizumab significantly reduced the blood eosinophil count in subjects who presented to the emergency department with a severe asthma exacerbation (WO 2013/066780). Benralizumab does not affect other cell lineages in the bone marrow or periphery. (Kolbeck R, et al. JACI 125:1344-53 (2010)).

Previous studies have demonstrated that an outpatient strategy focused on reducing eosinophils in the sputum reduces the number of subsequent asthma exacerbations (Green R, et al. Lancet 360:1715-21 (2002); Haldar P, et al. NEJM 360:973-84 (2009)). However, studies have not shown the effect of controlling asthma for patients with NP.

Thus, given the high unmet need of controlling asthma and that some subjects with asthma have nasal polyposis, the effect of benralizumab in adult subjects with asthma and nasal polyposis was examined.

### BRIEF SUMMARY

Methods of reducing the annual exacerbation rate of asthma in an asthma patient with nasal polyposis are provided herein. In certain aspects, a method of reducing the annual exacerbation rate of asthma in an asthma patient with nasal polyposis comprises administering to said asthma patient an effective amount of benralizumab or an antigen-binding fragment thereof.

Methods of treating asthma are also provided herein. In certain aspects, a method of treating asthma comprises administering to an asthma patient with nasal polyposis an effective amount of benralizumab or an antigen-binding fragment thereof, wherein the patient has a blood eosinophil count of at least 300 cells/μl prior to the administration.

In certain aspects, a method of treating asthma comprises administering to an asthma patient with nasal polyposis an effective amount of benralizumab or an antigen-binding fragment thereof, wherein the patient has a forced expiratory volume (FEV₁) of less than 80% predicted value prior to the administration.

In certain aspects, a method of treating asthma comprises administering at least two doses of benralizumab or an antigen-binding fragment thereof to an asthma patient with nasal polyposis.

In certain aspects of the methods provided herein, the administration reduces the patient's exacerbation rate. In certain aspects, the administration reduces the patient's annual exacerbation rate. In certain aspects, the annual exacerbations rate following administration of benralizumab or an antigen-binding fragment thereof is reduced by at least 40%. In certain aspects, the annual exacerbation rate following administration of benralizumab or an antigen-binding fragment thereof is reduced by at least 50%. In certain aspects, the annual exacerbation rate following administration of benralizumab or an antigen-binding fragment thereof is reduced by at least 60%. In certain aspects, the SNOT-22 score following administration of benralizumab or an antigen-binding fragment thereof is reduced by at least 7 points. In certain aspects, the SNOT-22 score following administration of benralizumab or an antigen-binding fragment thereof is reduced by at least 8 points.

In certain aspects of the methods provided herein, the asthma is eosinophilic asthma. In certain aspects, the patient has a blood eosinophil count of at least 300 cells/μl.

In certain aspects of the methods provided herein, the patient has a forced expiratory volume (FEV₁) of less than 80% predicted value prior to the administration. In certain aspects, the patient has an asthma control questionnaire score of at least 1.5 prior to the administration. In certain aspects, the patient uses high-dose inhaled corticosteroids (ICS). In certain aspects, the patient uses long-acting β2 agonists (LABA). In certain aspects, the patient has a history of exacerbations. In certain aspects, the history of exacerbations comprises at least two exacerbations in the year prior to the administration of benralizumab or an antigen-binding fragment thereof. In certain aspects, the history of exacerbations comprises no more than six exacerbations in the year prior to the administration of benralizumab or an antigen-binding fragment thereof

In certain aspects of the methods provided herein, at least two doses of benralizumab or an antigen-binding fragment thereof are administered to the patient.

In certain aspects, benralizumab or an antigen-binding fragment thereof is administered at about 30 mg per dose.

In certain aspects of the methods provided herein, benralizumab or an antigen-binding fragment thereof is administered once every four weeks to once every twelve weeks. In certain aspects, the benralizumab or antigen-binding fragment thereof is administered once every four weeks. In certain aspects, benralizumab or an antigen-binding fragment thereof is administered once every eight weeks. In certain aspects, benralizumab or an antigen-binding fragment thereof is administered once every four weeks for twelve weeks and then once every eight weeks.

In certain aspects of the methods provided herein, benralizumab or an antigen-binding fragment thereof is administered parenterally. In certain aspects, benralizumab or an antigen-binding fragment thereof is administered subcutaneously.

In certain aspects of the methods provided herein, benralizumab or an antigen-binding fragment thereof is administered in addition to corticosteroid therapy.

In certain aspects, a method of reducing the SNOT-22 score in an asthma patient with nasal polyposis comprises administering to said asthma patient 30 mg of benralizumab or an antigen-binding fragment thereof, wherein the patient has an blood eosinophil count of at least 300 cells/μl prior to the administration. In certain aspects, the 30 mg of benralizumab is administered once every four weeks. In certain aspects, the 30 mg of benralizumab is administered once every eight weeks. In certain aspects, the 30 mg of benralizumab is administered once every four weeks for twelve weeks and then once every eight weeks. In certain aspects, the administration is subcutaneous.

In certain aspects a method of treating asthma in patient with nasal polyposis, comprises administering to the patient 30 mg of benralizumab or an antigen-binding fragment thereof, wherein the patient has an blood eosinophil count of at least 150 cells/μl prior to the administration. In certain aspects, the 30 mg of benralizumab is administered once every four weeks. In certain aspects, the 30 mg of benralizumab is administered once every eight weeks. In certain aspects, the 30 mg of benralizumab is administered once every four weeks for twelve weeks and then once every eight weeks. In certain aspects, the administration is subcutaneous.

In certain aspects of the methods provided herein, the patient's St. George's Respiratory Questionnaire (SGRQ) score is reduced. In certain aspects of the methods provided herein, the patient's SNOT-22, SGRQ, and ACQ-6 scores are reduced, and the patient's FEV is increased. In certain aspects of the methods provided herein, the patient's SNOT-22 score is reduced by at least 8.9. In certain aspects of the methods provided herein, the patient's SGRQ score is reduced by at least 4 units, the patient's ACQ score is reduced by at least 0.5, the patient's FEV is increased by at least 200 mL, and/or the administration prevents asthma exacerbations for at least 24 weeks from the first administration. In certain aspects of the methods provided herein, the patient's SGRQ score is reduced by at least 4 units, the patient's ACQ score is reduced by at least 0.5, the patient's FEV is increased by at least 200 mL, and the administration prevents asthma exacerbations for at least 24 weeks from the first administration.

In certain aspects of the provided methods, administration of benralizumab or an antigen-binding fragment thereof results in the reduction in asthma exacerbation rates, St. George's Respiratory Questionnaire (SGRQ) Total Score, FEV₁, Asthma Control Questionnaire (ACQ-6) scores, and Sino-Nasal Outcome Test-22 (SNOT-22) scores, as shown in FIGS. 2-7.

In certain aspects of the provided methods, administration of benralizumab or an antigen-binding fragment thereof results in the reduction in in asthma exacerbation rates, St. George's Respiratory Questionnaire (SGRQ) Total Score, FEV₁, Asthma Control Questionnaire (ACQ-6) scores, and Sino-Nasal Outcome Test-22 (SNOT-22) scores as shown in Examples 1-2.

### BRIEF DESCRIPTION OF THE DRAWINGS/FIGURES

FIG. 1 shows the study flow diagram.

FIG. 2 shows the reduction in annualised AER for patients in the benralizumab group versus patients in the placebo group.

FIG. 3 shows the improvement from baseline in St. George's Respiratory Questionnaire (SGRQ) Total Score, FEV₁, and Asthma Control Questionnaire 6 (ACQ-6) for patients treated with benralizumab versus placebo.

FIG. 4 shows Clinician Global Impression of Change (CGI-C) and Patient Global Impression of Change (PGI-C) responders at end of treatment by improvement type. Responder defined as “very much improved” or “much improved” on the CGI-C or PGI-C.

FIG. 5 shows the improvements in Predominant Symptom and Impairment Assessment (PSIA) change from baseline based on top-ranked and average of top 3 ranked symptoms/impairments. Estimate of the mean change from baseline at each time point for PSIA for (A) top-ranked symptom/impairment and (B) average of top 3 ranked symptoms/impairments in the benralizumab group compared with the placebo group using a repeated measures analysis. Change from baseline in (A) top-ranked symptom/impairment and (B) average PSIA score of top 3 ranked symptoms/impairments.

FIG. 6 shows a forest plot of baseline factor effect on asthma exacerbation rate (AER), St. George's Respiratory Questionnaire (SGRQ) Total Score, Asthma Control Questionnaire 6 (ACQ-6), and FEV1 with benralizumab for the overall treatment population.

FIG. 7 shows the improvement from baseline in Sino-Nasal Outcome Test-22 (SNOT-22) for patients treated with benralizumab versus placebo (NP Substudy). Mean SNOT-22 total scores at baseline were similar for both treatment groups.

FIG. 8 shows the percent of patients that are responders based on increases in SNOT-22, Asthma exacerbation rate (AER), St. George's Respiratory Questionnaire (SGRQ), forced expiratory volume in 1 second (FEV₁), and asthma control questionnaire-6 (ACQ-6).

FIG. 9 shows the percent of patients with a comprehensive response.

### DETAILED DESCRIPTION

It is to be noted that the term “a” or “an” entity refers to one or more of that entity; for example, “an anti-IL-5α antibody” is understood to represent one or more anti-IL-5α antibodies. As such, the terms “a” (or “an”), “one or more,” and “at least one” can be used interchangeably herein.

Provided herein are methods for reducing exacerbations of asthma. The methods provided include administering an effective amount of benralizumab or an antigen-binding fragment thereof.

Information regarding benralizumab (or fragments thereof) for use in the methods provided herein can be found in U.S. Patent Application Publication No. US 2010/0291073 A1, the disclosure of which is incorporated herein by reference in its entirety. Benralizumab and antigen-binding fragments thereof for use in the methods provided herein comprise a heavy chain and a light chain or a heavy chain variable region and a light chain variable region. In a further aspect, benralizumab or an antigen-binding fragment thereof for use in the methods provided herein includes any one of the amino acid sequences of SEQ ID NOs: 1-4. In a specific aspect, benralizumab or an antigen-binding fragment thereof for use in the methods provided herein comprises a light chain variable region comprising the amino acid sequence of SEQ ID NO:1 and a heavy chain variable region comprising the amino acid sequence of SEQ ID NO:3. In a specific aspect, benralizumab or an antigen-binding fragment thereof for use in the methods provided herein comprises a light chain comprising the amino acid sequence of SEQ ID NO: 2 and heavy chain comprising the amino acid sequence of SEQ ID NO:4. In a specific aspect, benralizumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 7-9, and wherein the light chain variable region comprises the Kabat-defined CDR1, CDR2, and CDR3 sequences of SEQ ID NOs: 10-12. Those of ordinary skill in the art would easily be able to identify Chothia-defined, Abm-defined or other CDRs. In a specific aspect, benralizumab or an antigen-binding fragment thereof for use in the methods provided herein comprises the variable heavy chain and variable light chain CDR sequences of the KM1259 antibody as disclosed in U.S. Pat. No. 6,018,032, which is herein incorporated by reference in its entirety.

In certain aspects, a patient presenting at a physician's office or ED with asthma is administered benralizumab or an antigen-binding fragment thereof. Given the ability benralizumab to reduce or deplete eosinophil counts for up to 12 weeks or more (see US 2010/0291073), benralizumab or an antigen-binding fragment thereof can be administered only once or infrequently while still providing benefit to the patient in reducing exacerbations. In further aspects the patient is administered additional follow-on doses. Follow-on doses can be administered at various time intervals depending on the patient's age, weight, ability to comply with physician instructions, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. The intervals between doses can be every 4 weeks, every 5 weeks, every 6 weeks, every 8 weeks, every 10 weeks, every 12 weeks, or longer intervals. In certain aspects the intervals between doses can be every 4 weeks, every 8 weeks, or every 12 weeks. In certain aspects, the single dose or first dose is administered to the asthma patient shortly after the patient presents with an exacerbation, e.g., a mild, moderate or severe exacerbation. For example, benralizumab or an antigen-binding fragment thereof can be administered during a presenting clinic or hospital visit, or in the case of very severe exacerbations, within 1, 2, 3, 4, 5, 6, 7, or more days, e.g., 7 days of the acute exacerbation, allowing the patient's symptoms to stabilize prior to administration of benralizumab.

In some embodiments, at least two doses of benralizumab or an antigen-binding fragment thereof are administered to the patient. In some embodiments, at least three doses, at least four doses, at least five doses, at least six doses, or at least seven doses are administered to the patient. In some embodiments, benralizumab or an antigen-binding fragment thereof is administered over the course of four weeks, over the course of eight weeks, over the course of twelve weeks, over the course of twenty-four weeks, or over the course of a year.

The amount of benralizumab or antigen-binding fragment thereof to be administered to the patient will depend on various parameters such as the patient's age, weight, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. In certain aspects, the dosage or dosage interval is not dependent on the eosinophil level.

In certain aspects the patient is administered one or more doses of benralizumab or an antigen-binding fragment thereof, wherein the dose is about 2 mg to about 100 mg, for example about 20 mg to about 100 mg, or about 30 mg to about 100 mg. In certain specific aspects, the patient is administered one or more doses of benralizumab or an antigen-binding fragment thereof where the dose is about 20 mg, about 30 mg, about 40 mg, about 50 mg, about 60 mg, about 70 mg, about 80 mg, about 90 mg, or about 100 mg. In some embodiments, the dose is about 20 mg. In some embodiments the dose is about 30 mg. In some embodiments, the dose is about 100 mg.

In certain aspects, administration of benralizumab or an antigen-binding fragment thereof according to the methods provided herein is through parenteral administration. For example, benralizumab or an antigen-binding fragment thereof can be administered by intravenous infusion or by subcutaneous injection.

In certain aspects, benralizumab or an antigen-binding fragment thereof is administered according to the methods provided herein in combination or in conjunction with additional asthma therapies. Such therapies include, without limitation, inhaled corticosteroid therapy, long- or short-term bronchodilator treatment, oxygen supplementation, or other standard therapies as described, e.g., in the National Asthma Education and Prevention Program (NAEPP) Guidelines. In certain aspects, use of the methods provided herein, i.e., administration of benralizumab or an antigen-binding fragment thereof to an asthma patient with a history of exacerbations serves as adjunct therapy in situations of poor compliance with standard forms of asthma management.

The methods provided herein can significantly reduce exacerbations of asthma. Reduction can be measured based on the expected exacerbations predicted based on a large patient population, or based on the individual patient's history of exacerbations. In certain aspects, the patient population is those patients who had ≥2 exacerbations requiring systemic corticosteroid bursts in the past year. In certain aspects, the patient population is those patients who had ≥2 exacerbations requiring systemic corticosteroid bursts in the past year and ≤6 exacerbations requiring systemic corticosteroid bursts in the past year. In certain aspects, the patient population is patients having an eosinophil count of at least 300 cells/μl.

In certain aspects, use of the methods provided herein, i.e., administration of benralizumab or an antigen-binding fragment thereof reduces the number of exacerbations experienced by the patient over a 24-week period following administration of benralizumab or an antigen-binding fragment thereof, as compared to the number of exacerbations expected according to the patient's history, as compared to the average number of exacerbations expected in a comparable population of patients, or as compared to a comparable population treated with placebo over the same time period. In certain aspects, the patient can receive follow on doses of benralizumab or an antigen-binding fragment thereof at periodic intervals, e.g., every 4 weeks, every 5 weeks, every 6 weeks, every 8 weeks, every 12 weeks, or as scheduled based on patient's age, weight, ability to comply with physician instructions, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. Use of the methods provided herein can reduce the frequency of exacerbations by 10%, 20%, 30%, 35%, 40%, 45%, 50%, 55%, 60%, 65%, 70%, 75%, 80%, 85%, 90%, 95% or 100% over the 24-week period.

In other aspects, use of the methods provided herein, i.e., administration of benralizumab or an antigen-binding fragment thereof to an asthma patient, reduces the number of exacerbations experienced by the patient over a 52-week period (i.e., the annual exacerbation rate) following administration of benralizumab or an antigen-binding fragment thereof, as compared to the number of exacerbations expected according to the patient's history, as compared to the average number of exacerbations expected in a comparable population of patients, or as compared to a comparable population treated with placebo over the same time period. In certain aspects, the patient can receive follow on doses of benralizumab or an antigen-binding fragment thereof at periodic intervals, e.g., every 4 weeks, every 5 weeks, every 6 weeks, every 8 weeks, every 12 weeks, or as scheduled based on patient's age, weight, ability to comply with physician instructions, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. In certain aspects, the interval is every 4 weeks, every 8 weeks or every 12 weeks. Use of the methods provided herein can reduce the annual exacerbations by 10%, 20%, 30%, 35%, 40%, 45%, 50%, 55%, 60%, 65%, 70%, 75% , 80%, 85%, 90%, 95% or 100%.

In certain aspects, use of the methods provided herein, i.e., administration of benralizumab or an antigen-binding fragment thereof to an asthma patient, reduces the annual exacerbation rate, increases forced expiratory volume (FEV₁), improves an asthma questionnaire score (e.g., the asthma control questionnaire (ACQ)), improves the St. George's Respiratory Questionnaire (SGRQ) Total Score, and/or improves the Sino-Nasal Outcome Test-22 (SNOT-22) score.

In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points, and reduces the patient's asthma exacerbation rate (e.g., prevents an asthma exacerbation in the patient for at least 24 weeks from the first administration). In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points, and reduces the patient's SGRQ score, e.g., by at least 4 points. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points, and increases the patient's FEV, e.g., by at least 200 mL. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points, and reduces the patient's ACQ score, e.g., by at least 0.5 points.

In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; reduces the patient's ACQ score, e.g., by at least 0.5 points; and reduces the patient's SGRQ score, e.g., by at least 4 points. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; reduces the patient's ACQ score, e.g., by at least 0.5 points; and reduces the patient's asthma exacerbation rate (e.g., prevents an asthma exacerbation in the patient for at least 24 weeks from the first administration). In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; reduces the patient's SGRQ score, e.g., by at least 4 points; and reduces the patient's asthma exacerbation rate (e.g., prevents an asthma exacerbation in the patient for at least 24 weeks from the first administration). In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; increases the patient's FEV, e.g., by at least 200 mL; and reduces the patient's ACQ score, e.g., by at least 0.5 points. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; increases the patient's FEV, e.g., by at least 200 mL; and reduces the patient's SGRQ score, e.g., by at least 4 points. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; increases the patient's FEV, e.g., by at least 200 mL; and reduces the patient's asthma exacerbation rate (e.g., prevents an asthma exacerbation in the patient for at least 24 weeks from the first administration).

In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; reduces the patient's ACQ score, e.g., by at least 0.5 points; reduces the patient's SGRQ score, e.g., by at least 4 points; and reduces the patient's asthma exacerbation rate (e.g., prevents an asthma exacerbation in the patient for at least 24 weeks from the first administration). In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; increases the patient's FEV, e.g., by at least 200 mL; reduces the patient's ACQ score, e.g., by at least 0.5 points; and reduces the patient's SGRQ score, e.g., by at least 4 points. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; increases the patient's FEV, e.g., by at least 200 mL; reduces the patient's ACQ score, e.g., by at least 0.5 points; and reduces the patient's asthma exacerbation rate (e.g., prevents an asthma exacerbation in the patient for at least 24 weeks from the first administration). In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; increases the patient's FEV, e.g., by at least 200 mL; reduces the patient's SGRQ score, e.g., by at least 4 points; and reduces the patient's asthma exacerbation rate (e.g., prevents an asthma exacerbation in the patient for at least 24 weeks from the first administration).

In certain aspects, use of the methods provided herein, i.e., administration of benralizumab or an antigen-binding fragment thereof to an asthma patient (e.g., a patient with severe, eosinophilic asthma and with a history of nasal polyps), achieves a comprehensive response. In certain aspects, use of the methods provided herein, i.e., administration of benralizumab or an antigen-binding fragment thereof to an asthma patient (e.g., a patient with severe, eosinophilic asthma and with a history of nasal polyps), reduces the patient's SNOT-22 score, e.g., by at least 8.9 points; reduces the patient's ACQ score, e.g., by at least 0.5 points; reduces the patient's SGRQ score, e.g., by at least 4 points; reduces the patient's asthma exacerbation rate (e.g., prevents an asthma exacerbation in the patient for at least 24 weeks from the first administration); and increases the patient's FEV, e.g., by at least 200 mL.

In certain aspects, the patient is “eosinophilic positive” meaning the patient is one whose asthma is likely to be eosinophilic.

In certain aspects, the asthma patient has a particular blood eosinophil count, e.g., prior to the administration of benralizumab or an antigen-binding fragment thereof. Blood eosinophil counts can be measured, for example, using a complete blood count (CBC) with cell differential.

In certain aspects, the asthma patient has a blood eosinophil count of at least 300 cells/μl prior to the administration of benralizumab or an antigen-binding fragment thereof. In certain aspects, the asthma patient has a blood eosinophil count of at least 350 cells/μl, at least 400 cells/μl, at least 450 cells/μl, or at least 500 cells/μl prior to the administration of benralizumab or an antigen-binding fragment thereof.

In certain aspects, the asthma patient has a blood eosinophil count of less than 300 cells/μl prior to the administration of benralizumab or an antigen-binding fragment thereof. In certain aspects, the asthma patient has a blood eosinophil count of at least 100 cells/μl, at least 150 cells/μl, at least 180 cells/μl, at least 200 cells/μl, or at least 250 cells/μl prior to the administration of benralizumab or an antigen-binding fragment thereof.

In certain aspects, the asthma patient was prescribed or has been using a medium-dose of inhaled corticosteroids (ICS) use prior to the administration of benralizumab or an antigen-binding fragment thereof. A medium-dose of ICS can be a dose of at least 600 μg to 1,200 μg budesonide daily or an equivalent dose of another ICS.

In certain aspects, the asthma patient was prescribed or had been using a high-dose of ICS use prior to the administration of benralizumab or an antigen-binding fragment thereof. A high-dose of ICS can be a dose of at least 1,200 μg budesonide daily or an equivalent dose of another ICS. A high dose of ICS can also be a dose of greater than 1,200 μg to 2000 μg budesonide daily or an equivalent dose of another ICS.

In certain aspects, the asthma patient was prescribed or has been using oral corticosteroids prior to the administration of benralizumab or an antigen-binding fragment thereof. In certain aspects, administration of benralizumab or an antigen-binding fragment thereof decreases the use of oral corticosteroids in an asthma patient. In certain aspects, the administration decreases the use of oral corticosteroids in an asthma patient by at least 50%.

In certain aspects, the asthma patient was prescribed or had been using a long-acting beta agonist (LABA) prior to the administration of benralizumab or an antigen-binding fragment thereof.

In certain aspects, the asthma patient was prescribed or had been using both ICS and LABA prior to the administration of benralizumab or an antigen-binding fragment thereof.

In certain aspects, the asthma patient has a blood eosinophil count of at least 150 cells/μl.

In certain aspects, the asthma patient has a blood eosinophil count of at least 300 cells/μl and high ICS use prior to the administration of benralizumab or an antigen-binding fragment thereof.

In certain aspects, the asthma patient had a forced expiratory volume in 1 second (FEV₁) of at least 40% and less than 90% predicted value prior to the administration of benralizumab or an antigen-binding fragment thereof. In some embodiments, the FEV₁ was greater than 70% predicted value prior to the administration of benralizumab or an antigen-binding fragment thereof. In some embodiments, the FEV₁ was greater than 70% and less than 90% predicted value prior to the administration of benralizumab or an antigen-binding fragment thereof. In some embodiments, the FEV₁ was at least 75% predicted value prior to the administration of benralizumab or an antigen-binding fragment thereof. In some embodiments, the FEV₁ was at least 75% and less than 90% prior predicted value to the administration of benralizumab or an antigen-binding fragment thereof. In some embodiments, the FEV₁ was at least 80% predicted value prior to the administration of benralizumab or an antigen-binding fragment thereof. In some embodiments, the FEV₁ was at least 80% and less than 90% predicted value prior to the administration of benralizumab or an antigen-binding fragment thereof.

In certain aspects, the asthma patient has also been diagnosed with chronic sinusitis with nasal polyposis (NP) by a physician. SNOT-22 assesses the symptoms, sleep, and functional and emotional consequences of chronic rhinosinusitis with NP through responses to 22 items by using a 6-category scale from 0 (no problem) to 5 (problem as bad as it can be).

### EXAMPLES

### Example 1: Patients and Methods

### Subjects

Subjects in this study were required to be 18 to 75 years of age weighing at least 40 kg. They also must have had a physician diagnosis of asthma for a minimum of 12 months prior to screening as well as physician prescribed daily use of medium-dose or high-dose inhaled corticosteroids (ICS) plus another asthma controller (e.g., long-acting β₂ agonists (LABA), long-acting muscarinic antagonists (LAMA), leukotriene receptor antagonists, methylxanthines, or OCS) for at least 12 months prior to screening. Medium and high-doses of ICS as defined in this study are shown in Table 1 below.

TABLE 1
Estimated Comparative Daily Dosages for Inhaled Corticosteroids

| | Medium Daily | | High Daily | |
|---|---|---|---|---|
| Drug | Dose (Adult) | | Dose (Adult) | |
| Beclamethazone HFA/MDI | >240-480 | μg | >480 | μg |
| 40 or 801 μg/puff | | | | |
| Budesonide DPI | >600-1,200 | μg | >1,200 | μg |
| 90, 180, or 200 μg/inhalation | | | | |
| Ciclesonide HFA/MDI | >160-320 | μg | >320-1280 | μg |
| 80 or 160 μg/inhalation | | | | |
| Flunisolide CFC/MDI | >1,000-2,000 | μg | >2,000 | μg |
| 250 μg/puff | | | | |
| Flunisolide HFA/MDI | >320-640 | μg | >640 | μg |
| 80 μg/puff | | | | |
| Fluticasone | | | | |
| HFA/MDI: 44, 110, or 220 | >264-440 | μg | >440 | μg |
| μg/puff | | | | |
| DPI: 50, 100, or 250 μg/puff | >300-500 | μg | >500 | μg |
| Mometasone DPI | 400 | μg | >400 | μg |
| 200 μg/inhalation | | | | |
| Triamcinolone acetonide CFC/ | >750-1,500 | μg | >1,500 | μg |
| MDI 75 μg/puff | | | | |

CFC = chlorofluorocarbon; DPI = dry powder inhaler; HFA = hydrofluoroalkane; MDI = metered dose inhaler.

The dose of ICS and other asthma controller medications must have been stable in the subjects for at least 3 months prior to screening. Subjects must also have had at least 2 documented asthma exacerbations while on ICS plus another asthma controller that required treatment with systemic corticosteroids (IM, IV, or oral) in the 12 months prior to screening. Subjects must also have had a pre-bronchodilator forced expiratory volume in 1 second (FEV₁) of less than 80% predicted at Visit 2. Subjects must also have fulfilled one or more of the following criteria:
a. Airway reversibility (FEV₁≥12%) using a short-acting bronchodilator demonstrated at Visit 2 or Visit 3
b. Airway reversibility to short-acting bronchodilator (FEV₁≥12%) documented during the 12 months prior to enrolment Visit 1
c. Daily diurnal peak flow variability of >10% when averaged over 7 continuous days during the study run-in period
d. An increase in FEV₁ of ≥12% and 200 mL after a therapeutic trial of systemic corticosteroid (e.g., OCS), given outside an asthma exacerbation, documented in the 12 months prior to enrolment Visit 1
e. Airway hyper-responsiveness (methacholine: PC20 of <8 mg/mL, histamine: PD20 of <7.8 μmol, mannitol: decrease in FEV₁ as per the labelled product instructions) documented in the 24 months prior to randomisation Visit 4/Week 0

Subjects must also have had peripheral blood eosinophil count of either ≥300 cells/μL assessed by central laboratory at either Visit 1 or Visit 2, OR ≥150 to <300 cells/μL assessed by central laboratory at either Visit 1 or Visit 2, if ≥1 of the following 5 clinical criteria was met:
a) Using maintenance OCS (daily or every-other-day OCS requirement to maintain asthma control; maximum total daily dosage 20 mg prednisone or equivalent) at screening
b) History of nasal polyposis
c) Age of asthma onset
d) Three or more documented exacerbations requiring systemic corticosteroid treatment during the 12 months prior to screening
e) Pre-BD forced vital capacity <65% of predicted, as assessed at Visit 2 (note that screening pre-BD FEV₁ Inclusion Criterion above must still be satisfied).

Subjects must also have had an Asthma Control Questionnaire (ACQ) score of at least 1.5 at screening or during the screening/run-in period.

Subjects were not able to participate if they had a cigarette exposure of 10 pack-years or more or had been smoking within 12 months prior to screening or had any clinically important pulmonary condition other than asthma (e.g., active lung infection, chronic obstructive pulmonary disease [COPD], bronchiectasis, pulmonary fibrosis, cystic fibrosis), or ever been diagnosed with pulmonary or systemic disease, other than asthma, that was associated with elevated peripheral eosinophil counts (e.g., allergic bronchopulmonary aspergillosis/mycosis, ChurgStrauss syndrome, hypereosinophilic syndrome). Subjects were also not able to participate if they had previously received benralizumab or other concurrent biologics for asthma except for stable allergen immunotherapy, or systemic immunosuppressive medications within 3 months days prior to screening or during the screening/run-in period.

### Design of the Study

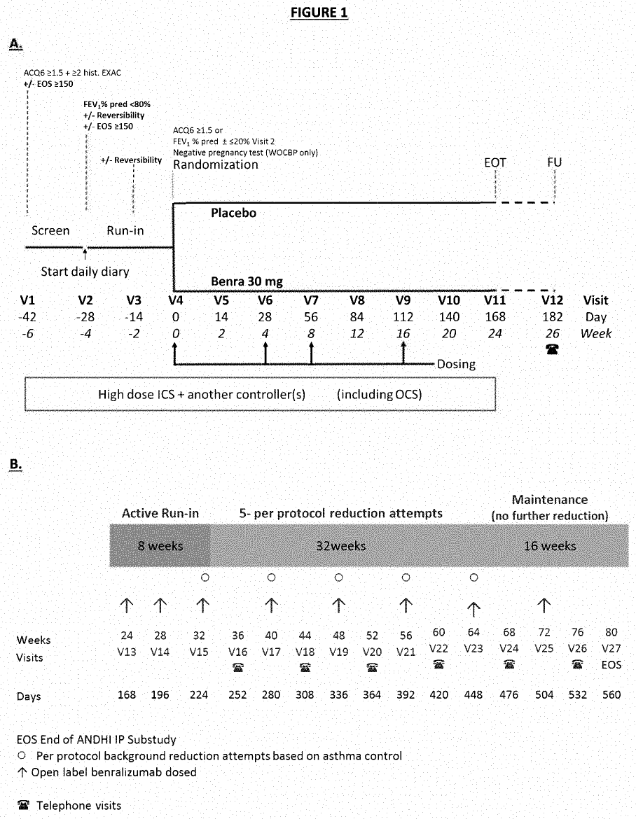

The ANDHI study was a phase 3b randomized, double-blind, placebo-controlled, dose-ranging, multicenter study (ClinicalTrials.gov number: NCT03170271) in which 30 mg of benralizumab were administered subcutaneously to asthma patients. The study flow diagram is shown in FIG. 1.

After enrolment at Visit 1, eligible patients entered an up to 42-day screening/run-in period. Patients who met eligibility criteria were randomly assigned on Visit 4 in a 2:1 ratio, stratified by prior exacerbation count (2/≥3), maintenance OCS use at Visit 1, and region, using an integrated voice recognition system/integrated web recognition system, to receive benralizumab 30 mg every 8 weeks (first three doses given 4 weeks apart) or matched placebo for 24 weeks. At the completion of the 24-week double-blind period of the study, eligible patients may enter a 56-week open label ANDHI IP substudy, in which concomitant asthma therapies will be tapered as directed by the protocol.
For those who transition directly into the open label ANDHI IP substudy, Visit 13 is on the same day as the ANDHI EOT Visit 11.
Patients who transition into the open label ANDHI IP substudy prior to FU Visit 12, will receive the first open label dose of benralizumab at Visit 13 and will complete the EOS visit at Week 80.
Those who do not enter the open label ANDHI IP substudy will have the FU visit 12 and then leave the study.
   Patients who completed the ANDHI FU Visit 12 are not excluded from participation in the ANDHI IP substudy.

Subjects received subcutaneous (SC) injections of 1 ml of benralizumab (30 mg/mL) or placebo for 4 doses: Day 0 (Week 0), Day 28 (Week 4), Day 56 (Week 8), and Day 112 (Week 16). In the open label ANDHI IP substudy, all eligible patients received benralizumab SC at Day 168 (Week 24), Day 196 (Week 28), Day 224 (Week 32), Day 280 (Week 40), Day 336 (Week 48), Day 392 (Week 56), Day 448 (Week 64), and Day 504 (Week 72).

Data were collected from all patients throughout the 24-week treatment period, which consisted of 8 study visits (Week 0/Visit 4, Week 2/Visit 5, Week 4/Visit 6, Week 8/Visit 7, Week 12/Visit 8, Week 16/Visit 9, Week 20/Visit 10, and Week 24/Visit 11). The planned baseline visit was Visit 4 for SGRQ, asthma symptom score (ACQ-6), pre-bronchodilator (pre-BD) FEV₁, CGI-C, PGI-C, and PSIA; Visit 3 was the planned baseline for SNOT-22. Baseline for daily diary measures was the average value over the 7 days prior to Visit 4.

Annualised AER was defined as total number of exacerbations×365.25/total duration of follow-up within the treatment group in days), which was compared across treatment groups over the 24-week treatment period. Time to first asthma exacerbation was analysed as a secondary efficacy variable. For the purpose of this study, an asthma exacerbation was defined as a worsening of asthma that led to any one of the following: use of systemic corticosteroids (or a temporary increase in a stable OCS background dosage) for at least 3 days; a single injectable dose of corticosteroids; an emergency room/urgent care visit (<24 hours) owing to asthma that required systemic corticosteroids; and an inpatient hospitalization (≥24 hours) because of asthma.

Change from baseline (Visit 4) to end of treatment (EOT) (Week 24/Visit 11) in SGRQ total score to determine the effect of benralizumab on patient-reported disease-specific HRQOL was also studied. The SGRQ is a 50-item patient-reported outcome (PRO) instrument developed to measure the health status of patients with airway obstruction diseases. The SGRQ total score indicates the impact of disease on overall health status and is expressed as a percentage of overall impairment (100 represents the worst possible health status and 0 indicates the best possible health status). A mean change score of 4 units on the SGRQ is associated with a minimum clinically important difference (MCID) and was used to assess SGRQ total score responder analysis at Weeks 4, 12, and 24.

FEV₁ was measured by spirometry at the study center. Asthma medication restrictions were to be followed before spirometry assessments were performed. All post randomisation spirometry assessments were performed within ±2 hours of the time at which the baseline pre-BD FEV₁ spirometry was performed. Patients measured their peak expiratory flow (PEF) using a peak flow meter each morning after awakening and before taking their morning asthma medications, as well as each evening. Change from baseline in weekly mean morning and mean evening PEF were each summarized and analyzed using a mixed-effect model repeated measure (MMRM).

To determine patient-reported asthma control, ACQ-6⁴⁸ was conducted to assess asthma symptoms. Questions (1 bronchodilator use question and 5 symptom questions) were weighted equally and scored from 0 (totally controlled) to 6 (severely uncontrolled), with individual change scores of at least 0.5 being considered clinically meaningful and was used for the responder analysis at Week 24; ACQ-6 scores of ≤0.75 indicate well-controlled asthma, scores between 0.75 and <1.5 indicate partly-controlled asthma, and a score ≥1.5 indicates uncontrolled asthma.

Clinician and Patient Global Impression of Change (CGI-C and PGI-C) assessments captured clinician and patient perception of change in disease-specific health status from baseline. The investigator (CGI-C) and the patient (PGI-C) rated the degree of change in overall asthma status compared with start of treatment at randomisation (Visit 4) using a 7-point rating scale (1 “very much improved;” 2 “much improved;” 3 “minimally improved;” 4 “no changes;” 5 “minimally worse;” 6 “much worse;” and 7 “very much worse”).

The Predominant Symptom and Impairment Assessment (PSIA) was developed for use in the study as a patient-driven assessment of impactful symptoms and impairments; given that this is the first use of the assessment, the measurement properties have not been established. As a PRO, the PSIA evaluated the degree to which patient-stated bothersome symptoms and impairments improved throughout the study. An individualised profile of symptoms and impairments, ranked by the patient in order of importance, was performed at Visit 3. Patients were presented a pre-specified list of 8 cardinal symptoms and impairments of asthma (shortness of breath, wheeze, cough, chest tightness, difficulty sleeping due to asthma, limited typical daily activities, limited physical intense activities, and sensitivity to environmental conditions) and asked to select those that impacted them over the past year. Patients then ranked the selected symptoms/impairments in order of impact from most impactful or top ranked (1) to least impactful (8). The PSIA was then individualised for each patient based on the top-ranked symptoms/impairments and administered throughout the study period. Patients were asked to report the severity of symptom/impairment over the previous 7 days on the individualised PSIA using an 11-point numeric rating scale from 0 (did not experience) to 10 (worst I can imagine).

SNOT-22 was used to determine the effect of benralizumab on disease-specific HRQOL for patients with physician-diagnosed chronic sinusitis with NP. SNOT-22 assesses the symptoms, sleep, and functional and emotional consequences of chronic rhinosinusitis with NP through responses to 22 items by using a 6-category scale from 0 (no problem) to 5 (problem as bad as it can be). The smallest change in the SNOT-22 that can be detected by a patient and associated with a MCID is 8.9.

### Safety Assessments

Adverse events were monitored following administration of placebo or benralizumab. Other assessments included physical examination, vital sign monitoring, and laboratory measurements.

### Example 2: Results

### Enrollment and Baseline Characteristics

The baseline characteristics of all randomized subjects are provided in Table 2 below. Demographics and baseline clinical characteristics were similar between both treatment groups, and the study population was representative of a patient population with severe, eosinophilic asthma (Table 2). The majority of patients were white (85.9%) and female (60.8%). The mean age was 52.8 years, and mean BMI was 29.94 kg/m². All patients reported exacerbations over the previous 12 months, with approximately half of the patients in each group (51.8% of benralizumab and 50.7% of placebo patients) experiencing 3 or more exacerbations. Lung function at screening, mean SGRQ total score, and mean ACQ-6 were also similar between groups. Mean PSIA severity scores at baseline for each of the top 3 ranked and for the average of the top 3 ranked impairments/symptoms were similar for both treatment groups.

Approximately 30% of patients in each group had BEC ≥150 to <300 cells/μL at screening. Overall, the greatest percentage of patients had a baseline BEC ≥450 cells/μL (41.9%), followed by <300 cells/μL (33.5%), and ≥300-<450 cells/μL (24.5%), and both treatment groups were balanced within each of these categories. Median baseline BEC was identical for both treatment groups (390 cells/μL).

The major categories of maintenance asthma medication used at baseline were generally balanced between groups. All patients were taking ICS and another asthma controller per inclusion criteria. Overall, 19.7% of patients were taking OCS and both groups were balanced for OCS use.

A total of 228 (34.8%) patients had a medical history of NP. Of these patients, 153 (23.3%) had NP at study entry and provided consent to be included into the NP substudy (96 and 57 patients randomised to benralizumab and placebo, respectively). For the 153 patients in the NP substudy analysis, mean SNOT-22 at baseline was 50.2, with similar mean SNOT-22 scores for patients in the benralizumab (51.5) and placebo (48.2) groups.

TABLE 2
Demographics and Baseline Clinical Characteristics

| | Benralizumab | | Placebo | |
|---|---|---|---|---|
| Demographic/Characteristic | (n = 427) | | (n = 229) | |
| Sex | 263 | (61.6) | 136 | (59.4) |
| Female, n (%) | | | | |
| Age (years) | 52.5 | (12.7) | 53.3 | (12.5) |
| Mean (SD) | | | | |
| Race | 314 | (86.0) | 168 | (85.7) |
| White, n (%) | | | | |
| BMI (kg/m²) | 29.85 | (7.37) | 30.10 | (7.89) |
| Mean (SD) | | | | |
| BEC group at screening, n (%) | | | | |
| ≥300 cells/μL | 297 | (69.7) | 165 | (72.4) |
| ≥150 to <300 cells/μL | 129 | (30.3) | 63 | (27.6) |
| BEC (cells/μL) at baseline | 390 | (40-7970) | 390 | (20-5600) |
| Median (range) | | | | |
| IgE values (IU/μL) | 139.65 | | 134.25 | |
| Median (range) | (1.5-6363.7) | | (1.5-11821.5) | |
| Phadiatop | 227 | (56.5) | 125 | (57.6) |
| Positive, n (%) | | | | |
| Exacerbations prior 12 months, | 3.2 | | 3.1 | |
| rate | | | | |
| 2, n (%) | 206 | (48.2) | 113 | (49.3) |
| ≥3, n (%) | 221 | (51.8) | 116 | (50.7) |
| SGRQ total score^{a} | 58.19 | (17.71) | 56.69 | (18.09) |
| Mean (SD) | | | | |
| Pre-BD FEV₁ | | | | |
| Mean (SD), mL | 1630 | (609) | 1720 | (629) |
| Percent-predicted normal (SD), | 54.0 | (14.2) | 55.9 | (13.6) |
| % | | | | |
| Post-BD FEV₁ | | | | |
| Mean (SD), mL | 2060 | (734) | 2110 | (727) |
| Percent-predicted normal (SD), | 68.0 | (16.44) | 68.6 | (15.24) |
| % | | | | |
| Reversibility | 28.2 | (20.43) | 24.9 | (19.15) |
| Mean (SD), % | | | | |
| ACQ-6^{a} | 3.04 | (0.874) | 3.07 | (0.965) |
| Mean (SD) | | | | |
| PSIA^{b} | | | | |
| Mean (SD) | | | | |
| Top-ranked symptom | 6.40 | (2.16) | 6.60 | (1.93) |
| impairment | | | | |
| Top 3 ranked symptoms/ | 6.16 | (1.82) | 6.32 | (1.85) |
| impairments | | | | |
| SNOT-22^{c} | 51.5 | (20.4) | 48.2 | (21.2) |
| Mean (SD) | | | | |

BEC = blood eosinophil counts; BMI = body mass index; FEV₁ = forced expiratory volume in 1 second; IgE = Immunoglobulin E; NP = nasal polyposis; SGRQ = St. George's Respiratory Questionnaire; SNOT-22 = Sino-Nasal Outcome Test-22.
^{a}Baseline measurement was the last non-missing assessment prior to or on the day of the first dose of study treatment.
^{b}Baseline measurement is the last non-missing assessment prior to the first dose of study treatment; if time is collected, the assessment performed the same day but at a time prior to the first dose of study treatment is included in baseline definition; if time is not collected, the assessment performed the same day is included in baseline definition.
^{c}Subgroup of patients providing consent to be included in the NP substudy for the SNOT-22 baseline: benralizumab (n = 96), placebo (n = 57).

### Efficacy

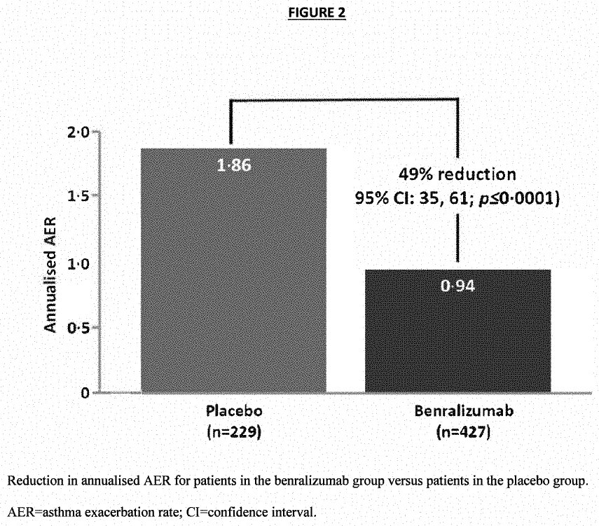

The effects of administration of benralizumab on exacerbation rates are shown in FIGS. 2-7. AER for patients treated with benralizumab was compared with placebo using a negative binomial model. The response variable in the model was the number of asthma exacerbations over the 24-week treatment period. The estimated treatment effect (i.e., the rate ratio [RR] of benralizumab vs placebo), corresponding 95% confidence interval (CI), and two-sided p-value for the RR were included. Time to first asthma exacerbation was analysed using a Cox proportional hazard model as a secondary efficacy variable to the primary objective with results presented as a hazard ratio (HR) and 95% CI Differences in least-squares (LS) mean change from baseline in SGRQ total score, FEV₁, and ACQ-6 at Week 24 for patients treated with benralizumab versus placebo were analysed. For SGRQ, FEV₁, and ACQ-6, analysis was via a MMRM with adjustment for treatment, baseline measure, region, number of exacerbations in previous year, maintenance OCS use at baseline visit, visit, and treatment×visit (for FEV₁ adjusted also for age and sex). For SGRQ, only the Week 24 comparison was controlled for multiplicity. ACQ-6 and FEV₁ were not multiplicity-controlled analyses, therefore all ACQ-6 and FEV₁ p-values are nominal. Responder analyses for SGRQ and ACQ-6 were analysed via a logistic regression model (adjusted for treatment, baseline score, region, number of exacerbations in the previous year, and baseline maintenance OCS use) with results reported as an odds ratio (OR) with associated 95% CI and nominal p-value.

Benralizumab significantly reduced annualized (annual) AER over the 24-week period compared with placebo by 49% in the overall population (RR estimate: 0.51; 95% CI: 0.39, 0.65) (FIG. 2). The treatment effect equated to a −0.92 difference in the annualized rate of exacerbations (AER) (p<0.0001). Time to first asthma exacerbation was longer for patients in the benralizumab group, as indicated by a 48% lower risk of having an asthma exacerbation compared with placebo (HR [95% CI]: 0.52 [0.40, 0.67]; p<0.0001). A total of 28.8% of patients in the benralizumab group versus 46.7% of patients in the placebo group reported asthma exacerbations from baseline through Week 24. For patients with baseline eosinophils ≥300 cells/μL, benralizumab significantly reduced AER over the 24-week period compared with placebo by 59% (RR [95% CI]: 0.41 [0.30, 0.56]).

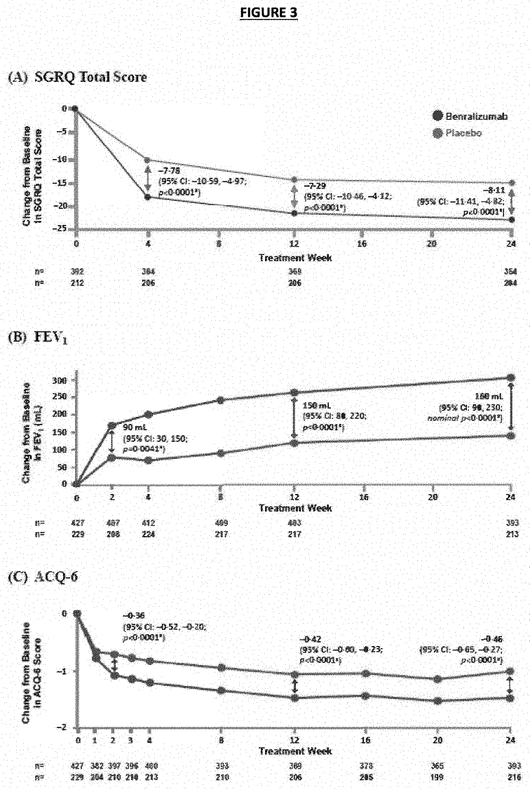

A clinically meaningful and statistically significant difference in LS mean change from baseline in SGRQ total score at Week 24 was observed for patients treated with benralizumab compared with placebo (−8.11; p≤0.0001), and those improvements were evident from Week 4 (first time point assessed) onward, with the greatest decrease seen at Week 24 (−23.06 units for benralizumab vs −14.94 units for placebo) (FIG. 3A). For patients with baseline eosinophils ≥300 cells/μL, a greater difference in LS mean change from baseline in SGRQ total score was demonstrated at Week 24 for benralizumab compared with placebo (−11.16). The percentage of patients with a clinically meaningful improvement in SGRQ total score (≥4 point decrease from baseline in total score) was consistently greater for the benralizumab group compared with the placebo group at all time points (Week 4: 70.3% vs 59.0%, Week 12: 70.5% vs 60.7%, Week 24: 72.01% vs 62.9%, respectively). Similarly, a lower percentage of patients in the benralizumab group reported a deterioration in their SGRQ total score ≥4 units during the treatment period compared with placebo (Week 4: 6.1% vs 17.5%, Week 12: 5.9% vs 13.1%, Week 24: 5.4% vs 14.0%, respectively). The likelihood of achieving a clinically meaningful improvement in SGRQ total score (MCID of 4 units) at EOT was greater for benralizumab-treated patients compared with placebo (80.1% vs 67.9%; OR: 1.91; [95% CI: 1.30, 2.81] p=0.0010).

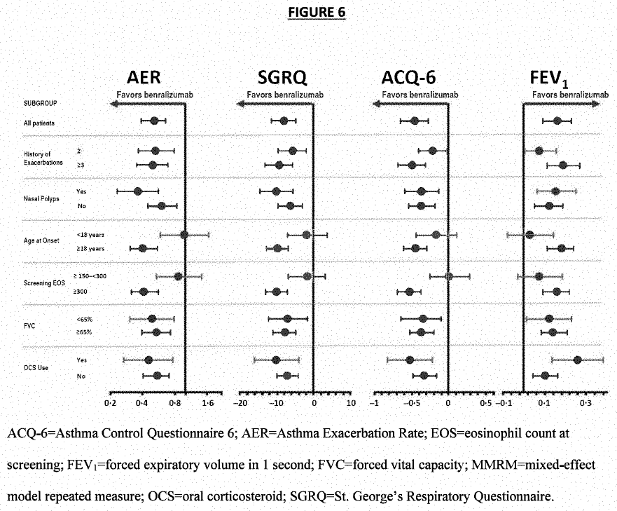

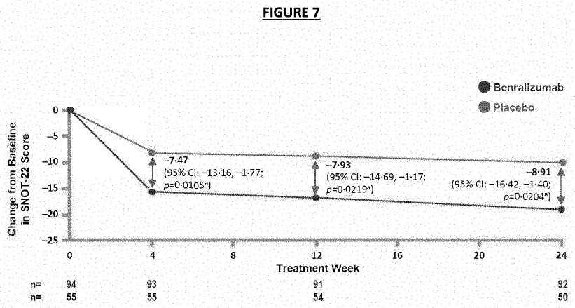

Benralizumab improved lung function at Week 24 versus placebo (LS mean difference: 160 mL [p<0.0001]), with improvements observed from the first time point assessed (Week 2 LS mean difference: 90 mL [p=0.0041]) onward (FIG. 3B). For patients with baseline eosinophils ≥300 cells/μL, a greater improvement in lung function versus placebo was demonstrated at week 24 (LS mean difference: 191 mL). The LS mean change from baseline in morning and evening PEF observed for the benralizumab group throughout the treatment period was greater than for the placebo group from Week 1 (p=0.0214 [morning]) and at all subsequent time points through Week 24 (p=0.0031 [morning]), indicating an early and sustained improvement. A comparison of the reduction in exacerbation rates in patients with less than 300 cells/μl and patients with at least 300 cells/μl prior to treatment is shown in FIG. 6, and the number of exacerbations at various eosinophil counts are provided in FIG. 7.

ACQ-6 score improvements were greater for the benralizumab group compared with the placebo group from Week 2 (LS mean difference: −0.36 units [p<0.0001]) through Week 24 (LS mean difference: −0.46 units [p<0.0001]), indicating an early and sustained improvement in ACQ-6 score throughout the treatment period (FIG. 3C). For patients with baseline eosinophils ≥300 cells/μL, a greater difference in LS mean change from baseline in ACQ-6 was demonstrated at Week 24 for benralizumab compared with placebo (−0.61). The likelihood of achieving a minimum clinically meaningful improvement in ACQ-6 score at EOT (MCID of ≤−0.5) was greater for patients treated with benralizumab (73.3%) compared with placebo (65.5%). There was a greater probability of achieving responder status per MCID at EOT in the benralizumab group compared with the placebo group (OR: 1.53; 95% CI: 1.07, 2.20; p=0.0193).

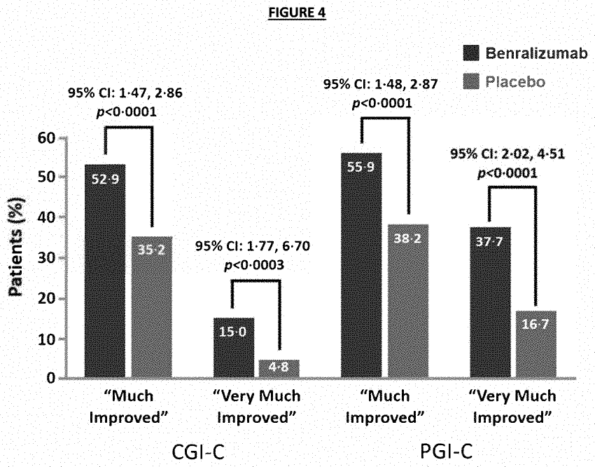

Assessment of perceived change from baseline showed a greater percentage of improved patients (“very much improved,” “much improved,” and “minimally improved”) in the benralizumab group throughout the treatment period compared with patients in the placebo group for CGI-C (Week 2: 57.6% vs 38.0%; Week 12: 63.9% vs 52.4%; Week 24: 67.7% vs 55.0%, respectively) and PGI-C (Week 2: 59.3% vs 41.9%; Week 12: 72.4% vs 5.5%; Week 24: 71.0% vs 58.1%, respectively). Patients tended to report more improvement on the PGI-C than clinicians reported on the CGI-C (data not shown). The likelihood of being a responder (defined as “very much improved” or “much improved” on the CGI-C or PGI-C for overall asthma status at the end of treatment [Week 24]) was greater for the benralizumab group compared with the placebo group for CGI-C (“very much improved” OR: 3.45; 95% CI: 1.77, 6.70; p=0.0003 and “much improved” OR: 2.05; 95% CI, 1.47, 2.86; p<0.0001) and PGI-C (“very much improved” OR: 3.02; 95% CI, 2.02, 4.51; p<0.0001 and “much improved” OR: 2.06; 95% CI 1.48, 2.87; p<0.0001) (FIG. 4).

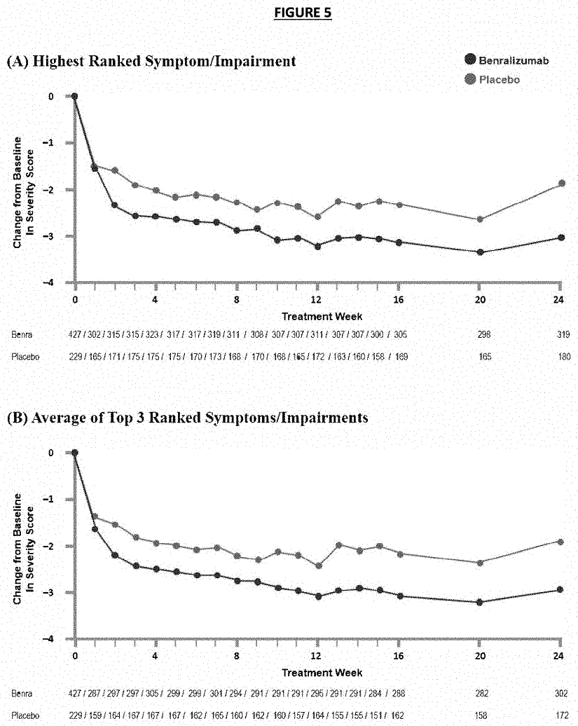

The benralizumab and placebo groups were similar in terms of the top ranked PSIA symptoms/impairments at initial assessment. Shortness of breath was the most commonly reported symptom/impairment for patients in the benralizumab and placebo groups (40.7% and 43.7% of patients, respectively), followed by limited physical intense activities (14.1% and 12.2%, respectively), cough (11.7% and 12.7%, respectively), and wheeze (9.8% and 10.9%, respectively) regardless of patient rank. Patients reported greater improvement on the symptom/impairment rated as most important (FIG. 5A) and the average of the top 3 symptoms/impairments (FIG. 5B) in the benralizumab group compared with the placebo group. Greater LS mean decreases from baseline were observed for the benralizumab group compared with the placebo group from Week 2 onward, demonstrating an early and sustained improvement in the symptoms that patients viewed as most impactful as captured by PSIA.

### Subgroup Analyses

Subanalyses of key endpoints to investigate the treatment effect within pre-defined subgroups, defined by the presence of specific clinical features associated with the asthma eosinophilic phenotype and/or enhanced benralizumab response, are depicted in FIG. 6. For AER, subgroup analyses indicated that eosinophils ≥300 cells/μL (interaction p-value p=0.0130), the presence of adult-onset asthma (p=0.0033) and a medical history of NP (p=0.0616) were associated with an enhanced treatment response (at a 10% significance level). Eosinophils ≥300 cells/uL (p=0.0056) and the presence of adult-onset asthma (p=0.0095) were also associated with an enhanced SGRQ response. Eosinophils ≥300 cells/uL (p=0.0020), adult-onset asthma (p=0.0676) and ≥3 exacerbations in the previous year (p=0.0376) were associated with an enhanced ACQ-6 response. Adult-onset asthma (p=0.0179), baseline OCS use (p=0.0264) and ≥3 exacerbations (p=0.0365) were associated with an enhanced FEV₁ response. For AER, SGRQ, and ACQ-6, the treatment effect in those with baseline OCS use was numerically greater than in the overall population, although not statistically significantly different from those without OCS use. Similarly, a medical history of NP and ≥3 exacerbations in the previous 12 months showed a numerically greater treatment effect in terms of SGRQ response, without statistical significance.

The subgroup analysis was repeated for the subpopulations of patients with screening BEC of ≥300 cells/μL (data not shown). Results were consistent with the main subgroup analyses.

### Nasal Polyposis Subgroup Analysis

Of the overall study population, 23% (153/656) participated in the NP substudy. Compared with the overall study population, the NP substudy population (n=96 benralizumab; n=57 placebo) had a lower percentage of female patients (42% in the NP substudy benralizumab group and 55% in the NP substudy placebo group vs approximately 60% in each treatment group in the full-study population) and greater baseline median BEC (approximately 500 cells/μL for patients in the NP substudy compared with 390 cells/μL in the full-study population).

Benralizumab patients demonstrated greater improvement from baseline in SNOT-22 total scores compared with placebo patients at Visit 11/Week 24 (−8.9 [p=0.0204]). Greater LS mean decreases from baseline in SNOT-22 total scores were seen beginning at the first time point assessed/Week 4 (−7.47 [p=0.0105]) to EOT for the benralizumab group compared with the placebo group, with the greatest LS mean decrease observed at Week 24 (FIG. 7).

### Safety

Adverse events (AEs) occurred at similar frequencies in patients treated with benralizumab and placebo. Most AEs reported were assessed as mild or moderate in intensity. No patients had an AE with an outcome of death.

### Discussion

This study demonstrates that benralizumab reduced exacerbation for eosinophilic asthma, particularly for patients with blood eosinophil counts ≥300 cells/μL for whom exacerbation reduction versus placebo was 59%. Additionally, benralizumab provided significant and clinically meaningful improvements in disease-specific HRQOL based on change in total SGRQ score beginning at the first post-baseline time point. This result supports the observation that SGRQ may be a more sensitive indicator of treatment effect for patients with severe, eosinophilic asthma compared with AQLQ(S)+12. Furthermore, benralizumab improves disease-specific HRQOL for patients with severe, eosinophilic asthma and NP of any severity, as demonstrated by the early and sustained improvement in SNOT-22. The treatment effect observed was clinically meaningful.

### Example 3

A post-hoc subanalysis of the previous study from Examples 1 and 2 was conducted to assess comprehensive response to benralizumab based on SNOT-22 and asthma measures. Patients with severe, eosinophilic asthma and a history of physician-diagnosed NP of any severity ongoing at baseline in Examples 1 and 2 were included in the post-hoc subgroup analysis to assess comprehensive response to benralizumab. Comprehensive response was defined as achieving a clinically meaningful improvement in SNOT-22 of −8.9 units and 4 additional criteria: 0 exacerbations, change from baseline to end of treatment (Week 24) in SGRQ total score of ≤−4 units, FEV₁ improvement of ≥200 mL, change from baseline to week 24 in ACQ-6 total score of ≤−0.5.

The baseline demographics of all subjects are provided in Table 3 below. Some differences were seen at baseline between treatment groups in asthma measures (i.e., exacerbation history, SGRQ, and FEV₁), but these differences were not statistically significant.

TABLE 3
Demographics and Baseline Clinical Characteristics for Patients
with Severe, Eosinophilic Asthma and Nasal Polyposis

| | Benralizumab | | Placebo | |
|---|---|---|---|---|
| Demographic/Characteristic | (N = 96) | | (N = 57) | |
| Sex | 53 | (55.2) | 24 | (42.1) |
| Female, n (%) | | | | |
| Age (years) | 53.1 | (12.3) | 52.6 | (11.1) |
| Mean (SD) | | | | |
| Race | 73 | (91.3) | 41 | (91.1) |
| White, n (%) | | | | |
| BMI (kg/m²) | 27.38 | (6.20) | 27.71 | (5.54) |
| Mean (SD) | | | | |
| BEC at baseline (cells/μl) | 515 | (90-7970) | 500 | (80-3900) |
| Median (range) | | | | |
| SNOT-22 | 51.5 | (20.4) | 48.2 | (21.2) |
| Mean (SD) | | | | |
| Exacerbations prior 12 | 3.4 | | 3.3 | |
| months, rate | | | | |
| 2, n (%) | 47 | (49.0) | 23 | (40.4) |
| ≥3, n (%) | 49 | (51.0) | 34 | (59.6) |
| Pre-BD FEV₁ | 1.7 | (0.61) | 1.92 | (0.71) |
| Mean (SD), L | | | | |
| Percentage predicted normal | 53.7 | (13.8) | 58.1 | (13.8) |
| (SD), % | | | | |
| SGRQ total score | 54.24 | (15.00) | 51.09 | (18.05) |
| Mean (SD) | | | | |
| ACQ-6 | 2.88 | (0.81) | 2.96 | (0.90) |
| Mean (SD) | | | | |

ACQ-6 = Asthma Control Questionnaire-6; BD = bronchodilator; BEC = blood eosinophil counts; BMI = body mass index; FEV₁ = forced expiratory volume in 1 second; SD = standard deviation; SGRQ = St. George's Respiratory Questionnaire; SNOT-22 = Sino-Nasal Outcome Test-22.

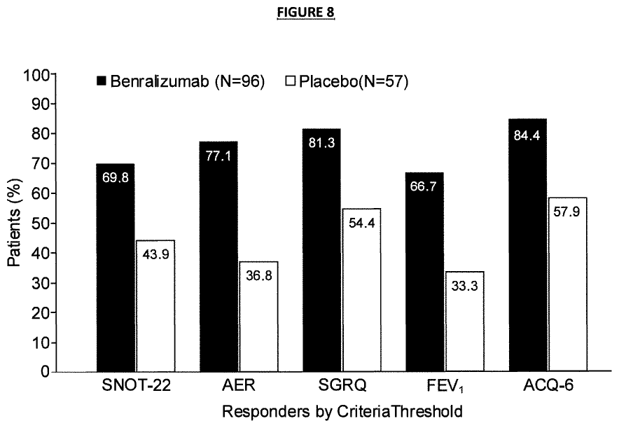

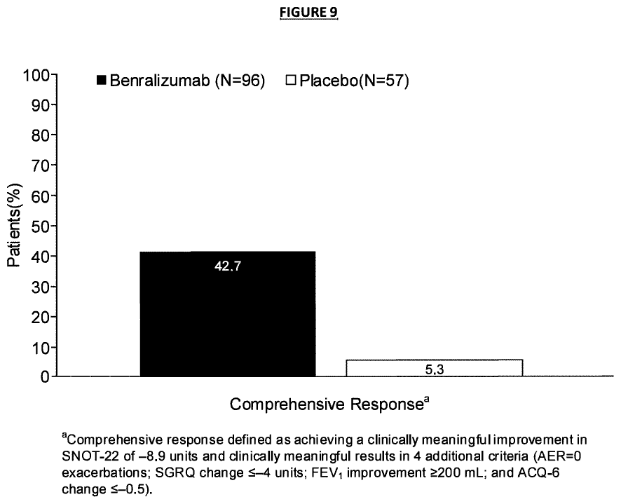

The percentage of patients with defined clinically meaningful improvements in each of the 5 endpoints at end of treatment (Week 24) was greater for benralizumab than placebo (FIG. 8). At week 24, comprehensive responders were more common with benralizumab (42.7%) vs. placebo (5.3%) (FIG. 9). The percentages of comprehensive responders based on fewer than 4 additional criteria increased for patients who met 3, 2, or 1 additional criteria (up to 53.1% vs. 12.3%, 60.4% vs. 24.6%, and 64.6% vs. 29.8% for benralizumab and placebo, respectively) (Table 4).

TABLE 4
Percentage of Comprehensive Responders Based on Fewer than 4
Additional Criteria: Benralizumab vs. Placebo^{a}

| | | |
|---|---|---|
| Demographic/Characteristic | Benralizumab (N = 96) | Placebo (N = 57) |

Clinically Meaningful Improvement in SNOT-22 and 3 Additional
Criteria

| | | |
|---|---|---|
| ACQ-6, SGRQ, and AER | 53.1% | 12.3% |
| FEV₁, ACQ-6, and SGRQ | 45.8% | 10.5% |
| FEV₁, ACQ-6, and AER | 43.8% | 5.3% |
| FEV₁, SGRQ, and AER | 42.7% | 5.3% |

Clinically Meaningful Improvement in SNOT-22 and 2 Additional
Criteria

| | | |
|---|---|---|
| ACQ-6 and SGRQ | 60.4% | 24.6% |
| ACQ-6 and AER | 55.2% | 12.3% |
| SGRQ and AER | 53.1% | 15.8% |
| FEV₁ and ACQ-6 | 49.0% | 14.0% |
| FEV₁ and SGRQ | 47.9% | 12.3% |
| FEV₁ and AER | 43.8% | 7.0% |

Clinically Meaningful Improvement in SNOT-22 and 1 Additional
Criterion

| | | |
|---|---|---|
| ACQ-6 | 64.6% | 29.8% |
| SGRQ | 62.5% | 29.8% |
| AER | 56.3% | 19.3% |
| FEV₁ | 52.1% | 17.5% |

ACQ-6 = Asthma Control Questionnaire-6; AER = asthma exacerbation rate; FEV₁ = forced expiratory volume in 1 second; SGRQ = St. George's Respiratory Questionnaire; SNOT-22 = Sino-Nasal Outcome Test-22.
^{a}Comprehensive response based on achieving a clinically meaningful improvement in SNOT-22 of −8.9 units and a clinically meaningful response in 3, 2, or 1 additional criteria (AER = 0 exacerbations; SGRQ change ≤−4 units; FEV₁ improvement ≥200 mL; and ACQ-6 change ≤−0.5).

The percentage of patients with the defined clinically meaningful improvements in each of the 5 endpoints at end of treatment was greater for benralizumab than placebo. The majority of patients with asthma and NP treated with benralizumab were SNOT-22 responders, as defined by a minimum clinically important difference of −8.9 points. Most patients with asthma and NP treated with benralizumab were comprehensive responders, achieving clinically meaningful improvement in SNOT-22 and multiple asthma outcomes (exacerbations, HRQOL, lung function, and asthma control). Comprehensive response was far more common for patients receiving benralizumab than placebo. Percentages of comprehensive responders based on fewer than 4 additional criteria increased for patients who met 3, 2, or 1 additional criteria, suggesting that these responses may not be completely correlated with one another.

Those skilled in the art will recognize, or be able to ascertain using no more than routine experimentation, many equivalents to the specific aspects of the disclosure described herein. Such equivalents are intended to be encompassed by the following claims.

Various publications are cited herein, the disclosures of which are incorporated by reference in their entireties.

Although the foregoing invention has been described in some detail by way of illustration and example for purposes of clarity of understanding, it will be obvious that certain changes and modifications can be practiced within the scope of the appended claims.

SEQUENCE LISTING

SEQ ID NO: 1
>US20100291073 1 Sequence 1 from Pat. U.S. 20100291073 Organism: Homo sapiens
DIQMTQSPSSLSASVGDRVTITCGTSEDIINYLNWYQQKPGKAPKLLIYHTSRLQSGVPSR
FSGSGSGTDFTLTISSLQP
EDFATYYCQQGYTLPYTFGQGTKVEIK
SEQ ID NO: 2
>US20100291073_2 Sequence 2 from Pat. U.S. 20100291073 Organism: Homo sapiens
DIQMTQSPSSLSASVGDRVTITCGTSEDIINYLNWYQQKPGKAPKLLIYHTSRLQSGVPSR
FSGSGSGTDFTLTISSLQP
EDFATYYCQQGYTLPYTFGQGTKVEIKRTVAAPSVFIFPPSDEQLKSGTASVVCLLNNFY
PREAKVQWKVDNALQSGNSQ
ESVTEQDSKDSTYSLSSTLTLSKADYEKHKVYACEVTHQGLSSPVTKSFNRGEC
SEQ ID NO: 3
>US20100291073_3 Sequence 3 from Pat. U.S. 20100291073 Organism: Homo sapiens
EVQLVQSGAEVKKPGASVKVSCKASGYTFTSYVIHWVRQRPGQGLAWMGYINPYNDG
TKYNERFKGKVTITSDRSTSTVY
MELSSLRSEDTAVYLCGREGIRYYGLLGDYWGQGTLVTVSS
SEQ ID NO: 4
>US20100291073_4 Sequence 4 from Pat. U.S. 20100291073 Organism: Homo sapiens
EVQLVQSGAEVKKPGASVKVSCKASGYTFTSYVIHWVRQRPGQGLAWMGYINPYNDG
TKYNERFKGKVTITSDRSTSTVY
MELSSLRSEDTAVYLCGREGIRYYGLLGDYWGQGTLVTVSSASTKGPSVFPLAPSSKSTS
GGTAALGCLVKDYFPEPVTV
SWNSGALTSGVHTFPAVLQSSGLYSLSSVVTVPSSSLGTQTYICNVNHKPSNTKVDKKVE
PKSCDKTHTCPPCPAPELLG
GPSVFLFPPKPKDTLMISRTPEVTCVVVDVSHEDPEVKFNWYVDGVEVHNAKTKPREEQ
YNSTYRVVSVLTVLHQDWLNG
KEYKCKVSNKALPAPIEKTISKAKGQPREPQVYTLPPSRDELTKNQVSLTCLVKGFYPSDI
AVEWESNGQPENNYKTTPP
VLDSDGSFFLYSKLTVDKSRWQQGNVFSCSVMHEALHNHYTQKSLSLSPGK
SEQ ID NO: 5
>US20100291073_5 Sequence 5 from Pat. US 20100291073 Organism: Homo sapiens
DLLPDEKISLLPPVNFTIKVTGLAQVLLQWKPNPDQEQRNVNLEYQVKINAPKEDDYET
RITESKCVTILHKGFSASVRT
ILQNDHSLLASSWASAELHAPPGSPGTSIVNLTCTTNTTEDNYSRLRSYQVSLHCTWLVG
TDAPEDTQYFLYYRYGSWTE
ECQEYSKDTLGRNIACWFPRTFILSKGRDWLAVLVNGSSKHSAIRPFDQLFALHAIDQINP
PLNVTAEIEGTRLSIQWEK
PVSAFPIHCFDYEVKIHNTRNGYLQIEKLMTNAFISIIDDLSKYDVQVRAAVSSMCREAGL
WSEWSQPIYVGNDEHKPLR
EWFVIVIMATICFILLILSLICKICHLWIKLFPPIPAPKSNIKDLFVTTNYEKAGSSETEIEVIC
YIEKPGVETLEDSVF
SEQ ID NO: 6
>US20100291073_6 Sequence 6 from Pat. US 20100291073 Organism: Mus musculus
DLLNHKKFLLLPPVNFTIKATGLAQVLLHWDPNPDQEQRHVDLEYHVKINAPQEDEYDT
RKTESKCVTPLHEGFAASVRT
ILKSSHTTLASSWVSAELKAPPGSPGTSVTNLTCTTHTVVSSHTHLRPYQVSLRCTWLVG
KDAPEDTQYFLYYRFGVLTE
KCQEYSRDALNRNTACWFPRTFINSKGFEQLAVHINGSSKRAAIKPFDQLFSPLAIDQVN
PPRNVTVEIESNSLYIQWEK
PLSAFPDHCFNYELKIYNTKNGHIQKEKLIANKFISKIDDVSTYSIQVRAAVSSPCRMPGR
WGEWSQPIYVGKERKSLVE
WHLIVLPTAACFVLLIFSLICRVCHLWTRLFPPVPAPKSNIKDLPVVTEYEKPSNETKIEVV
HCVEEVGFEVMGNSTF
SEQ ID NO: 7-VH CDR1
SYVIH
SEQ ID NO: 8-VH CDR2
YINPYNDGTKYNERFKG
SEQ ID NO: 9-VH CDR3
EGIRYYGLLGDY
SEQ ID NO: 10-VL CDR1
GTSEDIINYLN
SEQ ID NO: 11-VL CDR2
HTSRLQS
SEQ ID NO: 12-VL CDR3
QQGYTLPYT

What is claimed is:

1. A method of reducing the asthma exacerbation rate in an asthma patient with nasal polyposis, comprising administering to said asthma patient an effective amount of benralizumab or an antigen-binding fragment thereof, wherein the administration reduces the patient's exacerbation rate.

2. A method of treating asthma, comprising administering to an asthma patient with nasal polyposis an effective amount of benralizumab or an antigen-binding fragment thereof, wherein the patient has a blood eosinophil count of at least 300 cells/μl prior to the administration.

3. A method of treating asthma, comprising administering to an asthma patient with nasal polyposis an effective amount of benralizumab or an antigen-binding fragment thereof, wherein the patient has a forced expiratory volume (FEV₁) of less than 80% predicted value prior to the administration.

4. A method of treating asthma, comprising administering at least two doses of benralizumab or an antigen-binding fragment thereof to an asthma patient with nasal polyposis.

5. The method of any one of claims 2-4, wherein the administration reduces the patient's annual exacerbation rate.

6. The method of claim 5, wherein the administration improves the patient's Sino-Nasal Outcome Test-22 (SNOT-22) score.

7. The method of any one of claims 1 or 3-6, wherein the asthma is eosinophilic asthma.

8. The method of any one of claims 1 or 3-7, wherein the patient has a blood eosinophil count of at least 300 cells/μl.

9. The method of any one of claims 1, 2, or 4-8, wherein, the patient has a forced expiratory volume (FEV₁) of less than 75% predicted value prior to the administration.

10. The method of any one of claims 1-9, wherein the patient has an asthma control questionnaire 6 (ACQ-6) score of at least 1.5 prior to the administration.

11. The method of any one of claims 1-3 or 5-10, wherein at least two doses of benralizumab or an antigen-binding fragment thereof are administered to the patient.

12. The method of any one of claims 1 or 6-11, which reduces the annual exacerbations rate following administration of the antibody or antigen-binding fragment thereof.

13. The method of any one of claims 1 or 6-12, wherein the annual exacerbation rate is reduced by at least 40%.

14. The method of claim 13, wherein the annual exacerbation rate is reduced by at least 50%.

15. The method of claim 14, wherein the annual exacerbation rate is reduced by at least 60%.

16. The method of any one of claims 1-15, wherein the patient's SNOT-22 score is reduced by at least 7 points.

17. The method of any one of claims 1-16, wherein the patient's SNOT-22 score is reduced by at least 8 points.

18. The method of any one of claims 1-17, wherein the patient uses high-dose inhaled coritosteroids (ICS).

19. The method of any one of claims 1-18, wherein the patient uses long-acting β2 agonists (LABA).

20. The method of any one of claims 1-19, wherein the patient uses oral corticosteroids (OCS).

21. The method of any one of claims 1-20, wherein the patient has a history of exacerbations.

22. The method of claim 21, wherein the history of exacerbations comprises at least two exacerbations in the year prior to the administration of the benralizumab or antigen-binding fragment thereof.

23. The method of claim 21 or 22, wherein the history of exacerbations comprises no more than six exacerbations in the year prior to the administration of the benralizumab or antigen-binding fragment thereof.

24. The method of any one of claims 1-23, wherein the benralizumab or antigen-binding fragment thereof is administered at about 30 mg per dose.

25. The method of any one of claims 1-24, wherein the benralizumab or antigen-binding fragment thereof is administered once every four weeks to once every twelve weeks.

26. The method of claim 25, wherein the benralizumab or antigen-binding fragment thereof is administered once every four weeks.

27. The method of claim 25, wherein the benralizumab or antigen-binding fragment thereof is administered once every eight weeks.

28. The method of claim 25, wherein the benralizumab or antigen-binding fragment thereof is administered once every four weeks for twelve weeks and then once every eight weeks.

29. The method of any one of claims 1 to 28, wherein the benralizumab or antigen-binding fragment thereof is administered parenterally.

30. The method of claim 29, wherein the benralizumab or antigen-binding fragment thereof is administered subcutaneously.

31. The method of any one of claims 1 to 30, wherein the benralizumab or antigen-binding fragment thereof is administered in addition to corticosteroid therapy.

32. A method of reducing the SNOT-22 score in an asthma patient with nasal polyposis, comprising administering to an asthma patient 30 mg of benralizumab or an antigen-binding fragment thereof, wherein the patient has an blood eosinophil count of at least 300 cells/μl prior to the administration.

33. The method of claim 32, wherein the benralizumab or antigen-binding fragment thereof is administered once every four weeks for twelve weeks and then once every eight weeks.

34. The method of claim 32, wherein the benralizumab or antigen-binding fragment thereof is administered once every four weeks.

35. The method of claim 32, wherein the benralizumab or antigen-binding fragment thereof is administered once every eight weeks.

36. The method of any one of claims 32-35, wherein administration of the benralizumab or antigen-binding fragment decreases exacerbation rates of asthma.

37. The method of any one of claims 32-36, wherein administration of the benralizumab or antigen-binding fragment decreases annual exacerbation rates of asthma.

38. The method of any one of claims 32-36, wherein the administration is subcutaneous.

39. A method of treating asthma in patient with nasal polyposis, comprising administering the patient 30 mg of benralizumab or an antigen-binding fragment thereof, wherein the patient has an blood eosinophil count of at least 150 cells/μl prior to the administration.

40. The method of claim 39, wherein the benralizumab or antigen-binding fragment thereof is administered once every four weeks for twelve weeks and then once every eight weeks.

41. The method of claim 39, wherein the benralizumab or antigen-binding fragment thereof is administered once every four weeks.

42. The method of claim 39, wherein the benralizumab or antigen-binding fragment thereof is administered once every eight weeks.

43. The method of any one of claims 39-42, wherein the administration is subcutaneous.

44. The method of any one of claims 1-43, wherein the patient's St. George's Respiratory Questionnaire (SGRQ) score is reduced.

45. The method of any one of claims 1-44, wherein the patient's SNOT-22, SGRQ, and ACQ-6 scores are reduced, and the patient's FEV is increased.

46. The method of any one of claims 1-45, wherein the patient's SNOT-22 score is reduced by at least 8.9.

47. The method of any one of claims 1-46, wherein the patient's SGRQ score is reduced by at least 4 units, the patient's ACQ score is reduced by at least 0.5, the patient's FEV is increased by at least 200 mL, and/or the administration prevents asthma exacerbations for at least 24 weeks from the first administration.

48. The method of claim 47, wherein the patient's SGRQ score is reduced by at least 4 units, the patient's ACQ score is reduced by at least 0.5, the patient's FEV is increased by at least 200 mL, and the administration prevents asthma exacerbations for at least 24 weeks from the first administration.


SEQUENCE LISTING (12 sequences)

SEQ ID NO: 1 - protein, 107 aa, Homo sapiens [sequence omitted]

SEQ ID NO: 2 - protein, 214 aa, Homo sapiens
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 121 aa, Homo sapiens [sequence omitted]

SEQ ID NO: 4 - protein, 451 aa, Homo sapiens
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 400 aa, Homo sapiens
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 6 - protein, 398 aa, Mus musculus
DLLNHKKFLLLPPVNFTIKATGLAQVLLHWDPNPDQEQRHVDLEYHVKINAPQEDEYDTRKTESKCVTPLHEGFAASVRTILKSSHTTLASSWVSAELKAPPGSPGTSVTNLTCTTHTVVSSHTHLRPYQVSLRCTWLVGKDAPEDTQYFLYYRFGVLTEKCQEYSRDALNRNTACWFPRTFINSKGFEQLAVHINGSSKRAAIKPFDQLFSPLAIDQVNPPRNVTVEIESNSLYIQWEKPLSAFPDHCFNYELKIYNTKNGHIQKEKLIANKFISKIDDVSTYSIQVRAAVSSPCRMPGRWGEWSQPIYVGKERKSLVEWHLIVLPTAACFVLLIFSLICRVCHLWTRLFPPVPAPKSNIKDLPVVTEYEKPSNETKIEVVHCVEEVGFEVMGNSTF

SEQ ID NO: 7 - protein, 5 aa, Artificial Sequence
  Synthetic
SYVIH

SEQ ID NO: 8 - protein, 17 aa, Artificial Sequence
  Synthetic
YINPYNDGTKYNERFKG

SEQ ID NO: 9 - protein, 12 aa, Artificial Sequence
  Synthetic
EGIRYYGLLGDY

SEQ ID NO: 10 - protein, 11 aa, Artificial Sequence
  Synthetic
GTSEDIINYLN

SEQ ID NO: 11 - protein, 7 aa, Artificial Sequence
  Synthetic
HTSRLQS

SEQ ID NO: 12 - protein, 9 aa, Artificial Sequence
  Synthetic
QQGYTLPYT